(19) United States
(12) Patent Application Publication
(10) Pub. No.: US 2024/0197590 A1
(43) Pub. Date: Jun. 20, 2024

(54) CONDITIONING COMPOSITION FOR KERATIN FIBERS

(71) Applicant: KAO CORPORATION, Tokyo (JP)

(72) Inventors: Martin HOFFMANN, Darmstadt (DE); Ala-Mutka HELMI, Darmstadt (DE); Anne ENTZMINGER, Darmstadt (DE)

(73) Assignee: KAO CORPORATION, Tokyo (JP)

(21) Appl. No.: 18/526,401

(22) Filed: Dec. 1, 2023

(30) Foreign Application Priority Data
Dec. 2, 2022 (EP) 22211004.1

(51) Int. Cl. A61K 8/34; A45D 19/00; A61K 8/362; A61K 8/41; A61K 8/42; A61Q 5/12

(52) U.S. Cl. CPC A61K 8/345; A45D 19/005; A61K 8/342; A61K 8/362; A61K 8/416; A61K 8/42; A61Q 5/12; A45D 2019/0033

(57) ABSTRACT

The present invention is directed to a substantially anhydrous composition for keratin fibers especially for human hair comprising one or more polyol other than the glyceryl ether, one or more amine and/or quaternary ammonium surfactant, one or more fatty alcohol, one or more glyceryl ether according to the general structure

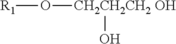

wherein R₁ is straight or branched, saturated or unsaturated alkyl chain with 4 to 18 C atoms, wherein the total concentration of water is not more than 10% by weight, calculated to the total weight of the composition, wherein the composition comprises one or more polyol at a concentration of at least 40% by weight, calculated to the total weight of the composition, and wherein the one or more glyceryl ether according to the above general structure is selected from ethylhexyl glycerin, hexylglycerin, heptylglycerin, methylheptylglycerin.

### CROSS REFERENCES TO RELATED APPLICATIONS

This application claims priority to European Patent Application No. 22211004.1, filed on Dec. 2, 2022, which is incorporated herein by reference in its entirety.

### FIELD OF THE INVENTION

The present invention is on a substantially anhydrous conditioning composition for keratin fibers, especially human hair which requires substantially less product application onto keratin fibers and therefore especially economical and has many advantageous properties with respect to the economics and environmental protection.

### BACKGROUND OF THE INVENTION

Hair conditioning compositions have been known and used for ages. They are used for detangling the hair and providing further additional properties. They are usually aqueous compositions based on cationic surfactants and may additionally comprise oily substances, polymers and others. They may be emulsions which are usually rinsed off after application and processing on the hair, and solutions and/or dispersions which are usually left on the hair and not rinsed off. Since the compositions are generally aqueous compositions, they comprise only certain amount of active and therefore requires relatively larger amount to be applied onto hair. This, furthermore, results in use of larger packaging materials mainly made of mixture of plastic materials which are often not recyclable and requires larger quantities to be transported which automatically means larger CO₂ emission. These compositions may be seen in several aspects uneconomical and environmentally disadvantageous. As clearly pointed out there are many aspects which should definitively be overcome for the sake of environment and to reduce or keep the promises that the temperature increase will not exceed 1.5° C. on average over time.

There have been efforts to overcome the above referred problems mainly by providing leave in compositions as detangling and further improvement of keratin fibers properties, which has not found a broad acceptance by the users because of mainly high loading on the hair, which results in volume reduction and makes hair in a way more covered and may as well be sticky.

Presence of cationic surfactants provide certain level of softening to the keratin fibers. It is quite important for the consumer feeling the softness during rinsing off the composition form hair and afterwards in towel dry and finally in dry state. Currently available conditioners have some drawback and therefore there is a need for further products.

WO 2021/219547 discloses substantially anhydrous compositions comprising one or more polyol, one or more mono alcohol, one or more fatty amine and one or more carboxylic acids. The compositions are transparent and provide certain level of conditioning on hair which has not always been found satisfactory.

### SUMMARY OF THE INVENTION

Accordingly, the first object of the present invention is a substantially anhydrous composition for keratin fibers especially for human hair comprising
one or more polyol other than the glyceryl ether,
one or more amine and/or quaternary ammonium surfactant,
one or more fatty alcohol,
one or more glyceryl ether according to the general structure

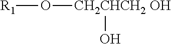

wherein R₁ is straight or branched, saturated or unsaturated alkyl chain with 4 to 18 C atoms,
wherein the composition comprises water not more than 10%, preferably 7.5%, more preferably 5% and most preferably 2.5% by weight, calculated to the total weight of the composition,
wherein the composition comprises one or more polyol at a concentration of at least 40% by weight, calculated to the total weight of the composition, and wherein the one or more glyceryl ether according to the above general structure is selected from ethylhexyl glycerin, hexylglycerin, heptylglycerin, methylheptylglycerin.

The term substantially anhydrous refers to the water content as given above. The composition comprises not more than 10% by weight of water. Preferably the composition does not comprise more than 7.5% by weight of water, more preferably not more than 5% by weight or water, and most preferably not more than 2.5% by weight of water, calculated to the total weight of the composition. The compositions are anhydrous in the particularly preferred form.

The second object of the present invention is a method of conditioning keratin fibers especially human hair, comprising the following steps:
(i) Treating the keratin fibers with an aqueous composition, preferably a cleansing composition, and then, when the keratin fibers are wet,
(ii) Applying the composition according to any of the claims 1 to 13 onto the keratin fibers and leaving it for a time period in the range of 30 s to 10 min, then
(iii) Rinsing off the composition from the keratin fibers, and then
(iv) Towel drying the keratin fibers.

The third object of the present invention is the use of the composition for conditioning keratin fibers, especially human hair, especially for improving softness, especially during application and rinsing off, surface smoothness, combability, shine and body.

Further object of the present invention is a method of conditioning keratin fibers especially human hair, comprising the following steps:
(i) Treating the keratin fibers with an aqueous composition, preferably a cleansing composition, and then, when the keratin fibers are wet,
(ii) Diluting the composition as defined above with water at a composition to water weight ratio of 1:1 to 1:10,
(iii) Applying the diluted composition onto the keratin fibers and leaving it for a time period in the range of 30 s to 10 min, then
(iv) Rinsing off the composition from the keratin fibers, and then
(v) Towel drying the keratin fibers.

Still further object of the present invention is a kit comprising two or more components wherein one of the components is a composition of the present invention.

Still another object of the present invention is a substantially anhydrous composition for keratin fibers comprising:
one or more polyol other than the glyceryl ether selected from glycerol,
one or more amine and/or quaternary ammonium surfactant,
one or more fatty alcohol,
one or more glyceryl ether according to the general structure

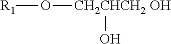

wherein R₁ is straight or branched, saturated or unsaturated alkyl chain with 4 to 18 C atoms,
wherein the total concentration of water is 10% by weight or less, calculated to the total weight of the composition,
wherein the composition comprises one or more polyol at a concentration of at least 60% by weight, calculated to the total weight of the composition, and
wherein the one or more glyceryl ether according to the above general structure is ethylhexyl glycerin.

### DETAILED DESCRIPTION OF THE INVENTION

The inventors of the present invention have surprisingly and unexpectedly found that a substantially anhydrous composition comprising one or more polyols, one or more amine or quaternary ammonium surfactants, one or more fatty alcohols and one or more glyceryl ethers provides good conditioning of the hair in terms of softness, especially during application and rinsing, i.e. improves the conditioning effect on wet hair such as surface smoothness, combability, shine and body, even when a considerably small amount of product is applied to the hair. The hair is rinsed after the product has been applied, evenly distributed and left on the hair for a short period of time, preferably from 30 seconds to 10 minutes, followed by towel-drying and air-drying or blow-drying the hair.

The composition of the present invention comprises one or more polyol other than glyceryl ether. The term polyol refers to liquid compounds having two or more OH groups in the molecule. The glyceryl ethers which additionally required for the present invention is not counted as the polyol. The one or more polyol comprised in the compositions at a total concentration of at least 40%, preferably at least 50%, more preferably in the range of 60 to 95% and most preferably in the range of 70% to 95%, by weight, calculated to the total composition.

The suitable non limiting examples are glycerin, 1,2-propylene glycol, 1,3 propane diol, 1,3-butylene glycol, 1,2-pentylene glycol, sorbitol, panthenol and mixtures thereof. Preferred are glycerin and 1,2-propylene glycol. The most preferred is glycerin.

The composition comprises one or more amine and/or quaternary ammonium surfactant. The suitable amine or quaternary ammonium surfactants are selected from the compounds according to the general structures A, B and C

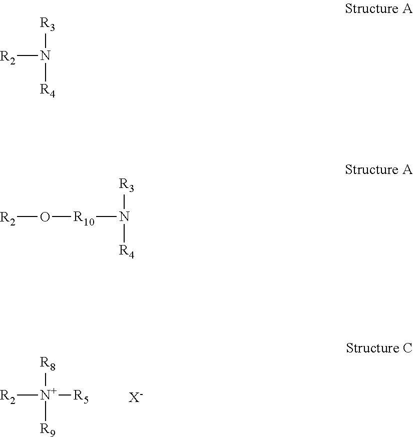

wherein R₂ is a saturated or unsaturated, branched, or straight alkyl chain with 8-24 C atoms or

R₆CONH(CH₂)ₙ
wherein R₆ is saturated or unsaturated, branched, or straight alkyl chain with 7-21 C atoms and n has value of 1-4, or

R₇COO(CH₂)ₙ
wherein R₇ is saturated or unsaturated, branched, or straight alkyl chain with 7-21 C atoms and n has value of 1-4,
wherein R₁₀ is a straight alkyl chain with 1-4 C atoms,
R₃ and R₄ are hydrogen or unsaturated or saturated, and straight or branched alkyl chain with 1-4 C atoms, and
R₈, R₉ and R₅ are unsaturated or saturated, branched, or straight alkyl chain with 1-4 C atoms,
wherein the terms unsaturated or saturated refers to C2 to C4 alkyl chain and branched alkyl chain refers to C3 to C4 alkyl chains.

Non-limiting suitable amines are such as stearyl amine, stearoylethyl amine, stearamidopropyldimethylamine, behenamidropyl dimethyl amine, stearyl amine, palmamine, oleamine, palmitamine, stearoxypropyl dimethylamine and non-limiting suitable quaternary ammonium compounds are such as cetrimonium chloride, steartrimonium chloride, behentrimonium chloride. Preferred are stearoxypropyl dimethylamine and cetrimonium chloride.

The one or more amine and/or quaternary ammonium compounds are comprised at a total concentration in the range of 0.1 to 10%, preferably 0.2 to 7.5% more preferably 0.25 to 5% by weight, calculated to the total composition.

The composition comprises one or more fatty alcohol. Preferably, one or more fatty alcohol is selected from the compounds according to the general structure

R₁₀—OH
wherein R₁₀ is a saturated or unsaturated, branched, or straight alkyl chain with 10 to 24C atoms.

Suitable non-limiting examples are cetyl alcohol, stearyl alcohol, cetearyl alcohol, behenyl alcohol, arachidyl alcohol, myristyl alcohol, isocetly alcohol and oleyl alcohol, and mixtures thereof.

The composition comprises one or more fatty alcohol at a total concentration in the range of 0.1 to 10%, preferably 0.2 to 7.5% more preferably 0.25 to 5% by weight calculated to the total composition.

The composition comprises one or more glyceryl ether according to the general structure presented above selected from ethylhexyl glycerin, hexylglycerin, heptylglycerin, and methylheptylglycerin. The most preferred glyceryl ether is ethylhexyl glycerin.

The composition comprises one or more glyceryl ether according to the general structure presented above at a total concentration in the range of 0.1 to 10%, preferably 0.2 to 7.5% more preferably 0.25 to 5% by weight, calculated to the total composition.

In an embodiment of the invention, the composition comprises one or more organic acids. The suitable non-limiting examples are citric acid, malic acid, lactic acid, glycolic acid, tartaric acid, formic acid, oxalic acid, malonic acid, succinic acid, glutaric acid, adipic acid, maleic acid, and fumaric acid. Preferred are citric acid, malic acid, lactic acid, succinic acid and maleic acid. The most preferred are citric acid, malic acid and lactic acid.

The one or more organic acid is comprised in the compositions at a concentration in the range of 0.01 to 2% by weight calculated to the total composition.

In a further embodiment of the present invention the composition comprises one or more linear and saturated mono alcohol having 2 to 6 carbon atoms. Suitable non-limiting examples are ethanol, butanol, propanol, pentanol, hexanol, and the one or more mono alcohol is comprised at a total concentration in the range of 1 to 30%, more preferably in the range of 2 to 25%, most preferably in the range of 5 to 25% by weight, calculated to the total composition.

The compositions of the present invention may be slightly viscous to pasty at room temperature. The compositions may be transparent or slightly cloudy to turbid. Suitably the compositions have a viscosity measured at 20° C. with Brookfield DVIII spindle 5/5 rpm after 30 sec in the range of 1000 to 30000 mPa·s. The compositions may be confectioned in a suitable bottle equipped with a nozzle; they may as well be packed in a bottle equipped with a dispenser.

The compositions have a pH in the range of 3.0 to 6.0, preferably 3.5 to 5.5 and more preferably 3.5 to 5.0 when diluted 1:3 by weight with water and at 20° C.

In principle there is no need to include preservatives in the compositions. Therefore, in an embodiment of the present invention the compositions comprises not more than 0.5%, preferably not more than 0.25% and most preferably not more than 0.1% antimicrobial preservatives.

The compositions may comprise any other compounds for other purposes such as fragrance, cationic polymers, ceramides, dyestuffs, thickening agents, oils including triglycerides, etc.

The following examples are to illustrate the invention but not to limit it.

### EXAMPLES

Example 1

| | Inv. | Comp. | Comp. | Comp. |
|---|---|---|---|---|
| | ex. 1 | ex. 1 | ex. 2 | ex. 3 |
| Ingredients | [% by weight] | | | |
| Ethylhexyl glycerin | 2.0 | — | — | 2.0 |
| Isostearyl glyceryl | — | — | 2.0 | — |
| ether | | | | |
| Cetrimonium chloride | 2.5 | 2.5 | 2.5 | 2.5 |
| Cetearyl alcohol | 4.0 | 4.0 | 4.0 | 4.0 |
| Citric acid | 0.05 | 0.05 | 0.05 | 0.05 |
| Fragrance | 0.5 | 0.5 | 0.5 | 0.5 |

| | | |
|---|---|---|
| Glycerol | ad 100.0 | — |
| C₁₃-C₁₅ alkane | — | ad 100.0 |

The above compositions were prepared by melting the fatty alcohol in glycerol or C₁₃-C₁₅ alkane at 75° C. and after cooling down to 40-45° C. temperature, the other compounds were added.

Comparative example 2 yielded a homogeneous composition immediately upon preparation, but during overnight storage the composition showed a phase separation, making the composition unsuitable for testing.

Comparative example 3 did not yield a homogeneous composition immediately upon preparation. Without being bound by any theory, the applicant believes that ethylhexyl glycerin and/or cetrimonium chloride were not soluble in C₁₃-C₁₅ alkane.

Inventive example 1 and comparative example 1 were tested as follows: In a half side test, the hair of 10 volunteers having approximately shoulder length hair and aging between 20 and 55 years was washed with a commercially available shampoo homogeneously and after dividing the hair on the head into two, the one side received the inventive composition, and the other side was applied the comparative composition. The amount applied was approximately 3 g composition each side.

After leaving the composition on the hair approximately 3 min it was rinsed off with water and towel dried. The hair was evaluated by hairdressers using the scale below.
1 not satisfied at all
2 little satisfied
3 satisfied
4 well satisfied
5 very well satisfied

The following average scores were obtained.

| | Comp. ex. 1 | Inv. ex. 1 |
|---|---|---|
| Wet hair | | |
| Easy to comb | 4 | 5 |
| Softness | 2 | 4 |
| Preference | 3 | 5 |
| Dry Hair | | |
| Easy to comb | 3 | 5 |
| Softness | 3 | 5 |
| Preference | 3 | 5 |

From the above, it is beyond any doubt the composition according to the invention has better conditioning effect on hair than the comparative composition.

Example 2

| | |
|---|---|
| Inv. ex. 2 | Comp. ex. 4 |

| | |
|---|---|
| Ingredients | [% by weight] |

| | | |
|---|---|---|
| Ethylhexyl glycerin | 2.0 | — |
| Behenamidopropyl | 2.5 | 2.5 |
| Dimethylamine | | |
| Cetearyl alcohol | 4.0 | 4.0 |
| Citric acid | 1.5 | 1.5 |
| Fragrance | 0.5 | 0.5 |

| | |
|---|---|
| Glycerol | ad 100.0 |

The above compositions were prepared by melting the fatty alcohol in glycerol at 75° C. and after cooling down to room temperature, the other compounds were added.

The half side test and the evaluation were done in the same way as in Example 1.

The following scores were obtained.

| | Comp. ex. 4 | Inv. ex. 2 |
|---|---|---|
| Wet hair | | |
| Easy to comb | 3 | 5 |
| Softness | 3 | 4 |
| Preference | 2 | 5 |
| Dry Hair | | |
| Easy to comb | 2 | 4 |
| Softness | 2 | 4 |
| Preference | 3 | 5 |

From the above, it is beyond any doubt the composition according to the invention has better conditioning effect on hair than the comparative composition.

Example 3

| | Inv. ex. 3 | Comp. ex. 4 | Comp. ex. 5 |
|---|---|---|---|
| Ingredients | [% by weight] | | |
| Ethylhexyl glycerin | 2.0 | — | 2.0 |
| Cetrimonium chloride | 2.5 | 2.5 | 2.5 |
| Cetearyl alcohol | 4.0 | 4.0 | 4.0 |
| Citric acid | 0.05 | 0.05 | 0.05 |
| Fragrance | 0.5 | 0.5 | 0.5 |
| Glycerol | 70.0 | 72.0 | — |
| Ethanol | Ad 100.0 | Ad 100.0 | — |
| Water | — | — | Ad 100.0 |

Inventive example 3 was prepared in the same way as Example 1. The composition had a good conditioning effect on human hair which was confirmed in a comparative test against a comparative example 4.

Comparative example 5 was prepared as an aqueous composition by melting the cetearyl alcohol in the presence of cetrimonium chloride in water and adding other substances afterwards. A comparative test was carried out against inventive example 1 as described under Example 1. Evaluation was also done in the same way as described under Example 1. The following results were obtained.

| | Comparative | Inventive |
|---|---|---|
| Wet hair | | |
| Easy to comb | 2 | 5 |
| Softness | 2 | 4 |
| Preference | 2 | 5 |
| Dry Hair | | |
| Easy to comb | 3 | 5 |
| Softness | 2 | 4 |
| Preference | 2 | 5 |

From the above results, it is beyond any doubt the anhydrous inventive example 3 conditioned hair much better than the above comparative example 5. It was especially pointed out by the testing of the hairdressers that the side with inventive example 3 was felt far softer than the side treated with the comparative example 5. The side treated with the inventive example was the preferred side and received the highest score.

1. A substantially anhydrous composition for keratin fibers especially for human hair comprising:
one or more polyol other than the glyceryl ether,
one or more amine and/or quaternary ammonium surfactant,
one or more fatty alcohol,
one or more glyceryl ether according to the general structure 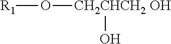
wherein R₁ is straight or branched, saturated or unsaturated alkyl chain with 4 to 18 C atoms,
wherein the total concentration of water is 10% by weight or less, calculated to the total weight of the composition,
wherein the composition comprises one or more polyol at a concentration of at least 40% by weight, calculated to the total weight of the composition, and
wherein the one or more glyceryl ether according to the above general structure is selected from ethylhexyl glycerin, hexylglycerin, heptylglycerin, methylheptylglycerin.

2. The composition according to claim 1, wherein the total concentration of water is 2.5% by weight or less, calculated to the total weight of the composition.

3. The composition according to claim 1, wherein the one or more amine and/or quaternary ammonium surfactant is selected from the compounds according to the following general structures A, B, or C: 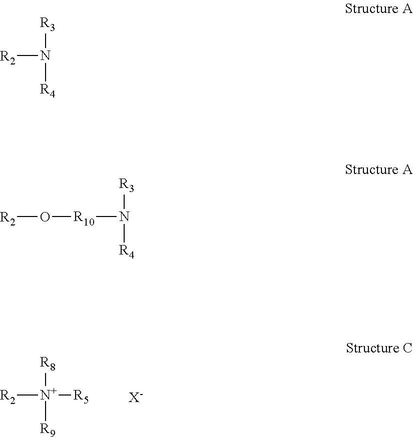
wherein R₂ is a saturated or unsaturated, branched, or straight alkyl chain with 8-24 C atoms or
R₆CONH (CH₂)ₙ
wherein R₆ is saturated or unsaturated, branched, or straight alkyl chain with 7-21 C atoms and n has value of 1-4, or
R₇COO(CH₂)ₙ
wherein R₇ is saturated or unsaturated, branched, or straight alkyl chain with 7-21 C atoms and n has value of 1-4,
wherein R₁₀ is a straight alkyl chain with 1-4 C atoms,
R₃ and R₄ are hydrogen or unsaturated or saturated, and straight or branched alkyl chain with 1-4 C atoms, and
R₈-, R₉ and R₅ are unsaturated or saturated, branched, or straight alkyl chain with 1-4 C atoms,
wherein the terms unsaturated or saturated refers to C2 to C4 alkyl chains and branched alkyl chain refers to C3 to C4 alkyl chains.

4. The composition according to claim 1, wherein the one or more fatty alcohol is selected from compounds according to the following general structure
R₁₀—OH
wherein R₁₀ is a saturated or unsaturated, branched, or straight alkyl chain with 10 to 24C atoms and preferably selected from cetyl alcohol, stearyl alcohol, cetearyl alcohol, behenyl alcohol, arachidyl alcohol, myristyl alcohol, isocetly alcohol and oleyl alcohol, and mixures thereof.

5. The composition according to claim 1, wherein the composition comprises one or more polyols at a concentration of at least 50% by weight, calculated to the total weight of the composition.

6. The composition according to claim 1, wherein the composition comprises one or more polyols at a concentration in the range of 60% to 90% by weight, calculated to the total weight of the composition.

7. The composition according to claim 1, wherein the one or more polyol is selected from glycerol, 1,2-propylene glycol, 1,3 propane diol, 1,3-butylene glycol, 1,2-pentylene glycol, sorbitol, panthenol, and mixtures thereof.

8. The composition according to claim 1, wherein the total concentration of the one or more amine or quaternary ammonium surfactants is in the range of 0.1% to 10% by weight, calculated to the total weight of the composition.

9. The composition according to claim 1, wherein the total concentration of the one or more amine or quaternary ammonium surfactants is in the range of 0.25% to 5% by weight, calculated to the total weight of the composition.

10. The composition according to claim 1, wherein the total concentration of the one or more fatty alcohols is in the range of 0.1% to 10% by weight, calculated to the total weight of the composition.

11. The composition according to claim 1, wherein the total concentration of the one or more glyceryl ether is in the range of 0.1% to 10% by weight, calculated to the total weight of the composition.

12. The composition according to claim 1, wherein the total concentration of the one or more glyceryl ether is in the range of 0.25% to 5% by weight, calculated to the total weight of the composition.

13. The composition according to claim 1, wherein the one or more glyceryl ether is ethylhexyl glycerin.

14. The composition according to claim 1, wherein the one or more amine or quaternary ammonium surfactant is selected from stearyl amine, stearoylethyl amine, stearamidopropyldimethylamine, behenamidropyl dimethyl amine, stearyl amine, palmamine, oleamine, palmitamine, stearoxypropyl dimethylamine, cetrimonium chloride, steartrimonium chloride, behentrimonium chloride, preferably said surfactant is selected from stearoxypropyl dimethylamine and cetrimonium chloride.

15. The composition according to claim 1, wherein the one or more amine or quaternary ammonium surfactant is selected from stearoxypropyl dimethylamine and cetrimonium chloride.

16. The composition according to claim 1, wherein the composition further comprises one or more organic acids selected from citric acid, malic acid, lactic acid, glycolic acid, tartaric acid, formic acid, oxalic acid, malonic acid, succinic acid, glutaric acid, adipic acid, maleic acid, and fumaric acid, and/or their mixtures.

17. The composition according to claim 1, wherein the composition has a pH in the range of 3.0 to 6.0, when diluted 1:3 by weight with water at 20° C.

18. The composition according to claim 1, wherein the composition further comprises one or more linear and saturated mono alcohol having 2 to 6 carbon atoms selected from ethanol, butanol, propanol, pentanol, hexanol.

19. A method of conditioning keratin fibers, comprising the following steps:
(i) Treating the keratin fibers with an aqueous composition, preferably a cleansing composition, and then, when the keratin fibers are wet,
(ii) Applying the composition according to claim 1 onto the keratin fibers and leaving it for a time period in the range of 30 s to 10 min, then
(iii) Rinsing off the composition from the keratin fibers, and then
(iv) Towel drying the keratin fibers.

20. A substantially anhydrous composition for keratin fibers comprising:
one or more polyol other than the glyceryl ether selected from glycerol,
one or more amine and/or quaternary ammonium surfactant,
one or more fatty alcohol,
one or more glyceryl ether according to the general structure 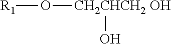
wherein R₁ is straight or branched, saturated or unsaturated alkyl chain with 4 to 18 C atoms,
wherein the total concentration of water is 10% by weight or less, calculated to the total weight of the composition,
wherein the composition comprises one or more polyol at a concentration of at least 60% by weight, calculated to the total weight of the composition, and
wherein the one or more glyceryl ether according to the above general structure is ethylhexyl glycerin.
